(19) United States
(12) Patent Application Publication
(10) Pub. No.: US 2009/0123538 A1
(43) Pub. Date: May 14, 2009

(54) Angiotensin II Receptor Antagonists

(76) Inventors: Laman L. Alani, Lansdale, PA (US); David C. Dubost, Collegeville, PA (US); Bruce S. Foster, Scotch Plains, NJ (US); Soumojeet Ghosh, Lansdale, PA (US); Hossain Jahansouz, North Wales, PA (US); Nazaneen Pourkavoos, Ambler, PA (US); Bhagwant Rege, Collegeville, PA (US); Aditya Tatavarti, Lansdale, PA (US)

Correspondence Address: MERCK AND CO., INC, P O BOX 2000, RAHWAY, NJ, 07065-0907, US

(21) Appl. No.: 11/886,553

(22) PCT Filed: Apr. 14, 2006
(86) PCT No.: PCT/US2006/014092

§ 371 Date: Sep. 17, 2007

Related U.S. Application Data
(60) Provisional application No. 60/673,086, filed on Apr. 20, 2005.

(51) Int. Cl. A61K 9/20; C07D 257/04; A61K 31/41; A61P 9/12; A61K 9/16

(52) U.S. Cl. 424/464; 548/250; 424/497; 424/494; 514/381

(57) ABSTRACT

The compounds of the present invention are polymorphic crystalline forms of the compound 2-butyl-4-chloro-1-[(2′-(1-H-tetrazol-5-yl)biphenyl-4-yl)methyl]-imidazole-5-carboxylic acid, which has the structure (I). Specifically, the compounds of the invention are selected from the group consisting of 2-butyl-4-chloro-1-[(2′-(1-H-tetrazol-5-yl)biphenyl-4-yl)methyl]-imidazole-5-carboxylic acid of Claim 3 selected from the group consisting of 2-butyl-4-chloro-1-[(2′-(1-H-tetrazol-5-yl)biphenyl-4-yl)methyl]-imidazole-5-carboxylic acid hydrochloride Form I, 2-butyl-4-chloro-1-[(2′-(1-H-tetrazol-5-yl)biphenyl-4-yl)methyl]-imidazole-5-carboxylic acid hydrochloride Form II, 2-butyl-4-chloro-1-[(2′-(1-H-tetrazol-5-yl)biphenyl-4-yl)methyl]-imidazole-5-carboxylic acid hydrochloride Form III, and 2-butyl-4-chloro-1-[(2′-(1-H-tetrazol-5-yl)biphenyl-4-yl)methyl]-imidazole-5-carboxylic acid monohydrate Form IV, 2-butyl-4-chloro-1-[(2′-(1-H-tetrazol-5-yl)biphenyl-4-yl)methyl]-imidazole-5-carboxylic acid monohydrate Form V, 2-butyl-4-chloro-1-[(2′-(1-H-tetrazol-5-yl)biphenyl-4-yl)methyl]-imidazole-5-carboxylic acid monohydrate Form VI, 2-butyl-4-chloro-1-[(2′-(1-H-tetrazol-5-yl)biphenyl-4-yl)methyl]-imidazole-5-carboxylic acid monohydrate Form VII, and 2-butyl-4-chloro-1-[(2′-(1-H-tetrazol-5-yl)biphenyl-4-yl)methyl]-imidazole-5-carboxylic acid monohydrate Form VIII.

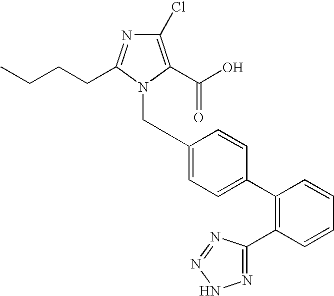

### BACKGROUND OF THE INVENTION

U.S. Pat. No. 5,136,069 generically and specifically describes 2-butyl-4-chloro-1-[p-(o-1H-tetrazol-5-ylphenyl)-benzyl]imidazole-5-methanol potassium salt and the anhydrous form of 2-butyl-4-chloro-1-[(2′-(1-H-tetrazol-5-yl)biphenyl-4-yl)methyl]-imidazole-5-carboxylic acid hydrochloride. Columns 261-263 of U.S. Pat. No. 5,136,069 describe general procedures for formulating compounds described in the patent, including capsules, tablets, injection formulations, and suspensions. U.S. Pat. No. 5,153,197, describes the use of these compounds, alone and in combination with a diuretic, to treat a patient having hypertension. U.S. Pat. No. 5,310,929 describes 5-imidazole carboxylic ester angiotensin II receptor antagonist prodrugs.

2-Butyl-4-chloro-1-[(2′-(1-H-tetrazol-5-yl)biphenyl-4-yl)methyl]imidazole-5-carboxylic acid is the active metabolite of 2-butyl-4-chloro-1-[p-(o-1H-tetrazol-5-ylphenyl)benzyl]imidazole-5-methanol potassium salt (also known as losartan potassium salt).

### SUMMARY OF THE INVENTION

The present invention includes angiotensin II receptor antagonist polymorphic forms of 2-butyl-4-chloro-1-[(2′-(1-H-tetrazol-5-yl)biphenyl-4-yl)methyl]-imidazole-5-carboxylic acid, including various pharmaceutically acceptable salts and hydrates of these forms, and pharmaceutical formulations for controlled and sustained delivery of these forms to a patient.

The salts of the polymorphic forms of 2-butyl-4-chloro-1-[(2′-(1-H-tetrazol-5-yl)biphenyl-4-yl)methyl]-imidazole-5-carboxylic acid of the invention include non-toxic salts such as those derived from inorganic acids, e.g. hydrochloric, hydrobromoic, sulfuric, sulfamic, phosphoric, nitric and the like, or the quaternary ammonium salts which are formed, e.g., from inorganic or organic acids or bases. Examples of acid addition salts include acetate, adipate, alginate, aspartate, benzoate, benzenesulfonate, bisulfate, butyrate, citrate, camphorate, camphorsulfonate, cyclopentanepropionate, digluconate, dodecylsulfate, ethanesulfonate, fumarate, glucoheptanoate, glycerophosphate, hemisulfate, heptanoate, hexanoate, hydrochloride, hydrobromide, hydroiodide, 2-hydroxyethanesulfonate, lactate, maleate, methanesulfonate, 2-naphthalenesulfonate, nicotinate, nitrate, oxalate, pamoate, pectinate, persulfate, 3-phenylpropionate, picrate, pivalate, propionate, succinate, sulfate, tartrate, thiocyanate, tosylate, and undecanoate. Base salts include ammonium salts, alkali metal salts such as sodium and potassium salts, alkaline earth metal salts such as calcium and magnesium salts, salts with organic bases such as dicyclohexylamine salts, N-methyl-D-glucamine, and salts with amino acids such as arginine, lysine, and so forth. Also, the basic nitrogen-containing groups may be quaternized with such agents as lower alkyl halides, such as methyl, ethyl, propyl, and butyl chloride, bromides and iodides; dialkyl sulfates like dimethyl, diethyl, dibutyl; and diamyl sulfates, long chain halides such as decyl, lauryl, myristyl and stearyl chlorides, bromides and iodides, aralkyl halides like benzyl and phenethyl bromides and others.

The invention includes an angiotensin II receptor antagonist polymorphic form selected from the group consisting of 2-butyl-4-chloro-1-[(2′-(1-H-tetrazol-5-yl)biphenyl-4-yl)methyl]-imidazole-5-carboxylic acid hydrochloride Form I, 2-butyl-4-chloro-1-[(2′-(1-H-tetrazol-5-yl)biphenyl-4-yl)methyl]-imidazole-5-carboxylic acid hydrochloride Form II, 2-butyl-4-chloro-1-[(2′-(1-H-tetrazol-5-yl)biphenyl-4-yl)methyl]-imidazole-5-carboxylic acid hydrochloride Form III, 2-butyl-4-chloro-1-[(2′-(1-H-tetrazol-5-yl)biphenyl-4-yl)methyl]-imidazole-5-carboxylic acid monohydrate Form IV, 2-butyl-4-chloro-1-[(2′-(1-H-tetrazol-5-yl)biphenyl-4-yl)methyl]-imidazole-5-carboxylic acid monohydrate Form V, 2-butyl-4-chloro-1-[(2′-(1-H-tetrazol-5-yl)biphenyl-4-yl)methyl]-imidazole-5-carboxylic acid monohydrate Form VI, 2-butyl-4-chloro-1-[(2′-(1-H-tetrazol-5-yl)biphenyl-4-yl)methyl]-imidazole-5-carboxylic acid monohydrate Form VII, 2-butyl-4-chloro-1-[(2′-(1-H-tetrazol-5-yl)biphenyl-4-yl)methyl]-imidazole-5-carboxylic acid monohydrate Form VIII.

The invention also includes a method for treating hypertension, congestive heart failure, pulmonary hypertension, renal insufficiency, renal ischemia, renal failure, renal fibrosis, cardiac insufficiency, cardiac hypertrophy, cardiac fibrosis, myocardial ischemia, cardiomyopathy, glomerulonephritis, renal colic, complications resulting from diabetes such as nephropathy, vasculopathy and neuropathy, glaucoma, elevated intra-ocular pressure, atherosclerosis, restenosis post angioplasty, complications following vascular or cardiac surgery, erectile dysfunction, hyperaldosteronism, lung fibrosis, scleroderma, anxiety, cognitive disorders, complications of treatments with immunosuppressive agents, and other diseases known to be related to the renin-angiotensin system, by administering an angiotensin II receptor antagonist polymorphic form of the invention to a patient having one or more of these conditions.

### DESCRIPTION OF THE DRAWINGS

FIG. 1 is the X-ray powder diffraction pattern for 2-butyl-4-chloro-1-[(2′-(1-H-tetrazol-5-yl)biphenyl-4-yl)methyl]-imidazole-5-carboxylic acid hydrochloride Form I.

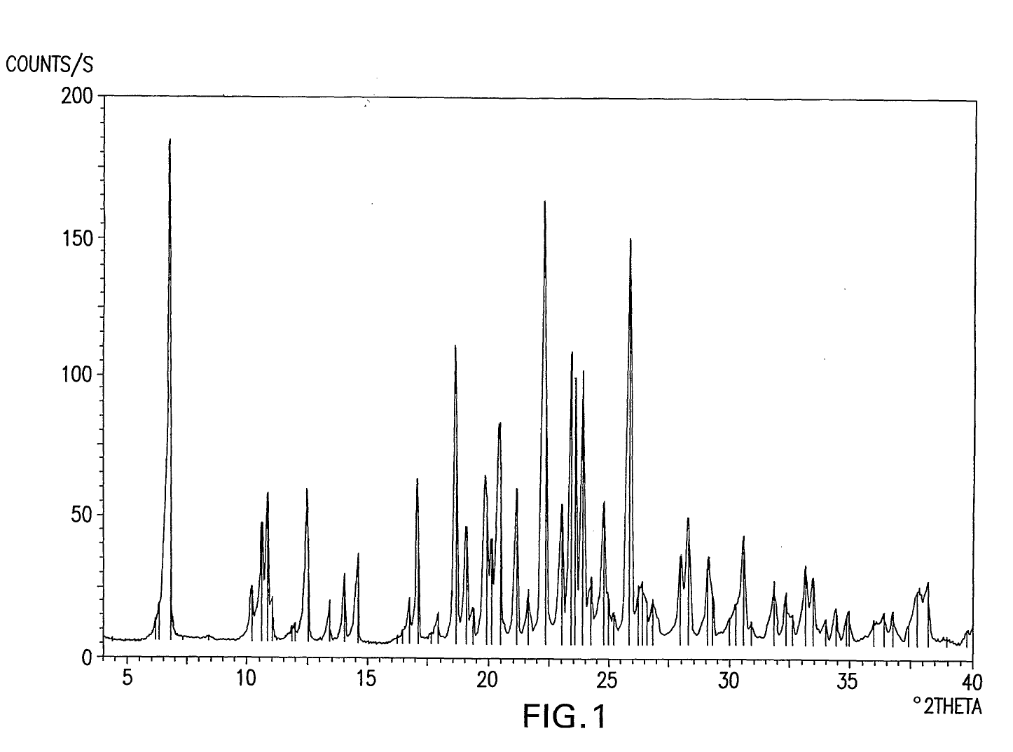

FIG. 2 is the X-ray powder diffraction pattern for 2-butyl-4-chloro-1-[(2′-(1-H-tetrazol-5-yl)biphenyl-4-yl)methyl]-imidazole-5-carboxylic acid hydrochloride Form II.

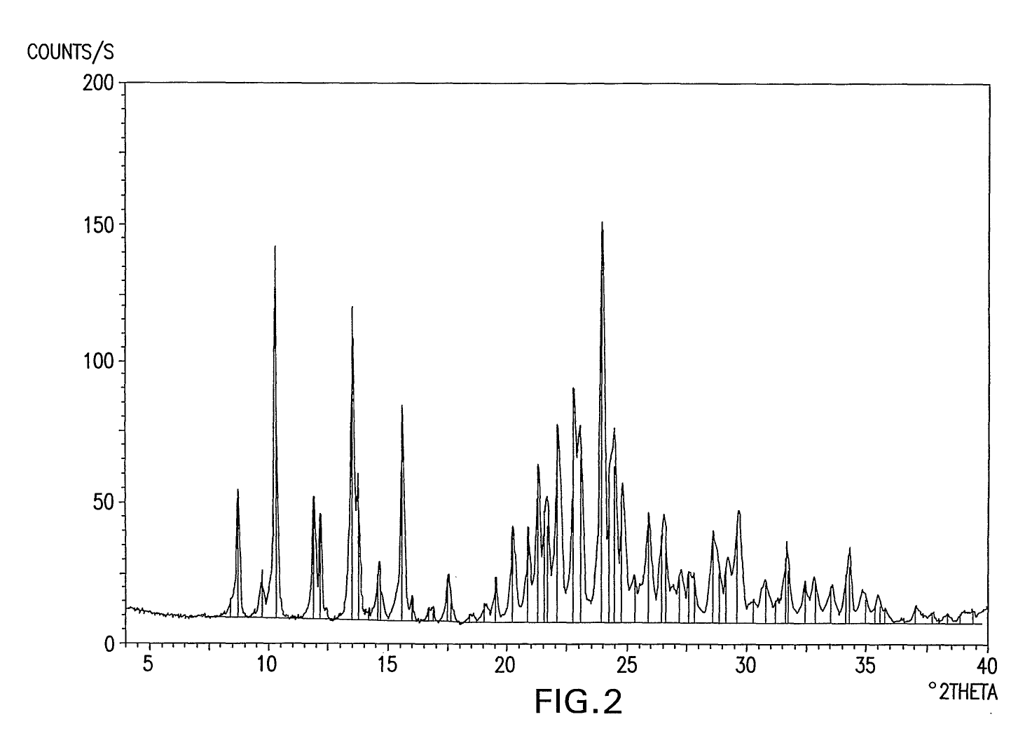

FIG. 3 is the X-ray powder diffraction pattern for 2-butyl-4-chloro-1-[(2′-(1-H-tetrazol-5-yl)biphenyl-4-yl)methyl]-imidazole-5-carboxylic acid hydrochloride Form III.

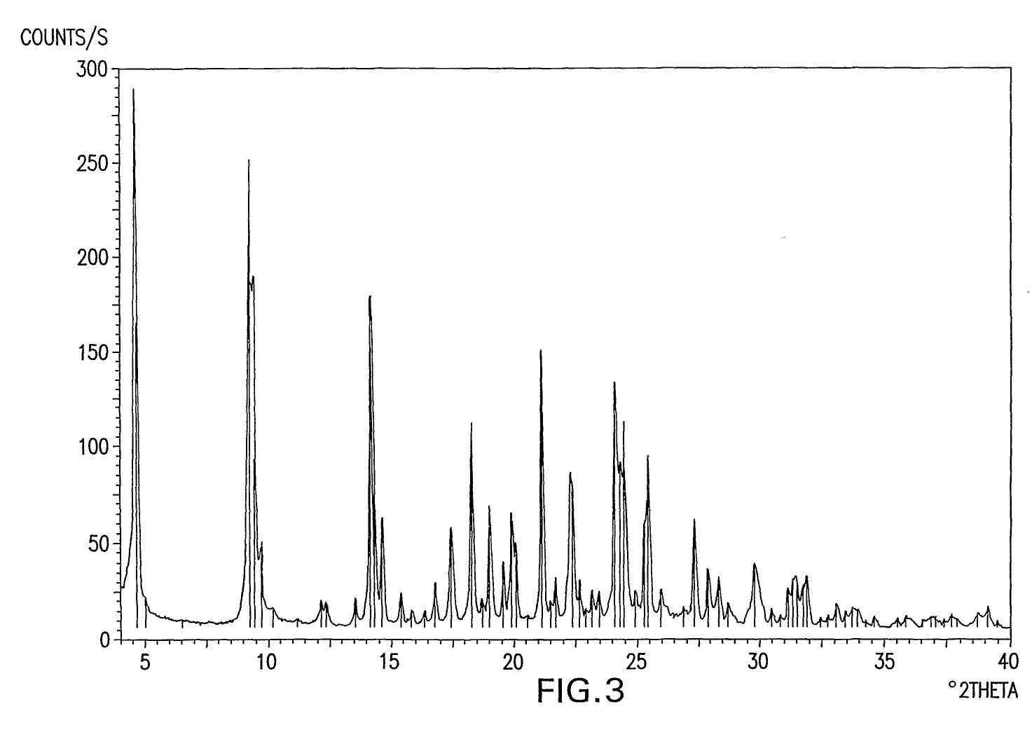

FIG. 4 is the X-ray powder diffraction pattern for 2-butyl-4-chloro-1-[(2′-(1-H-tetrazol-5-yl)biphenyl-4-yl)methyl]-imidazole-5-carboxylic acid monohydrate Form IV.

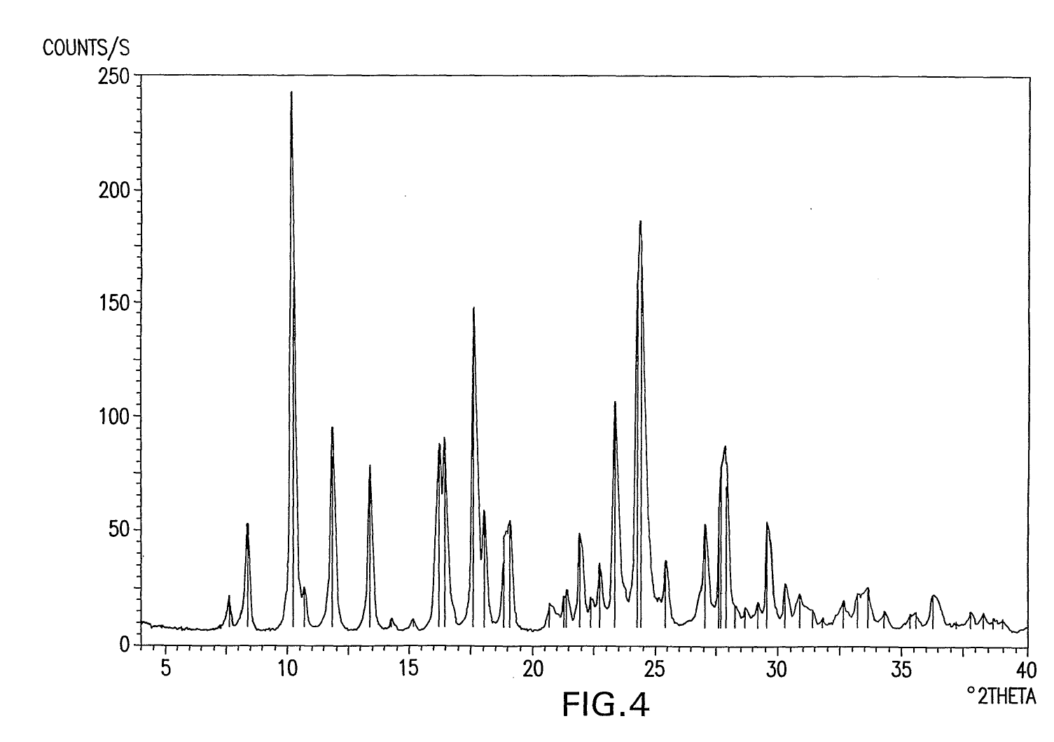

FIG. 5 is the X-ray powder diffraction pattern for 2-butyl-4-chloro-1-[(2′-(1-H-tetrazol-5-yl)biphenyl-4-yl)methyl]-imidazole-5-carboxylic acid monohydrate Form V.

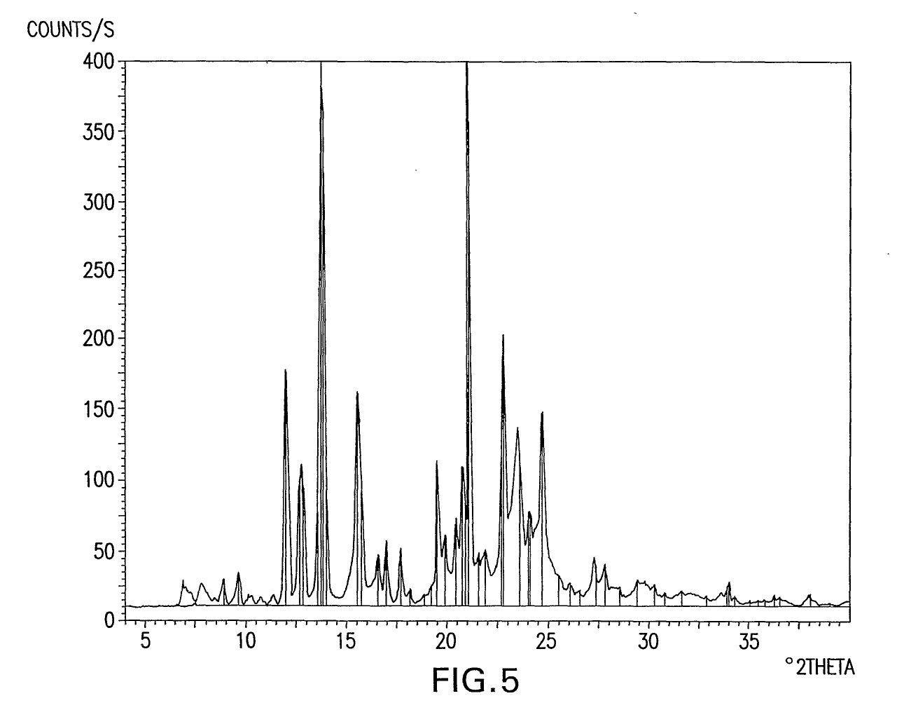

FIG. 6 is the X-ray powder diffraction pattern for 2-butyl-4-chloro-1-[(2′-(1-H-tetrazol-5-yl)biphenyl-4-yl)methyl]-imidazole-5-carboxylic acid monohydrate Form VI.

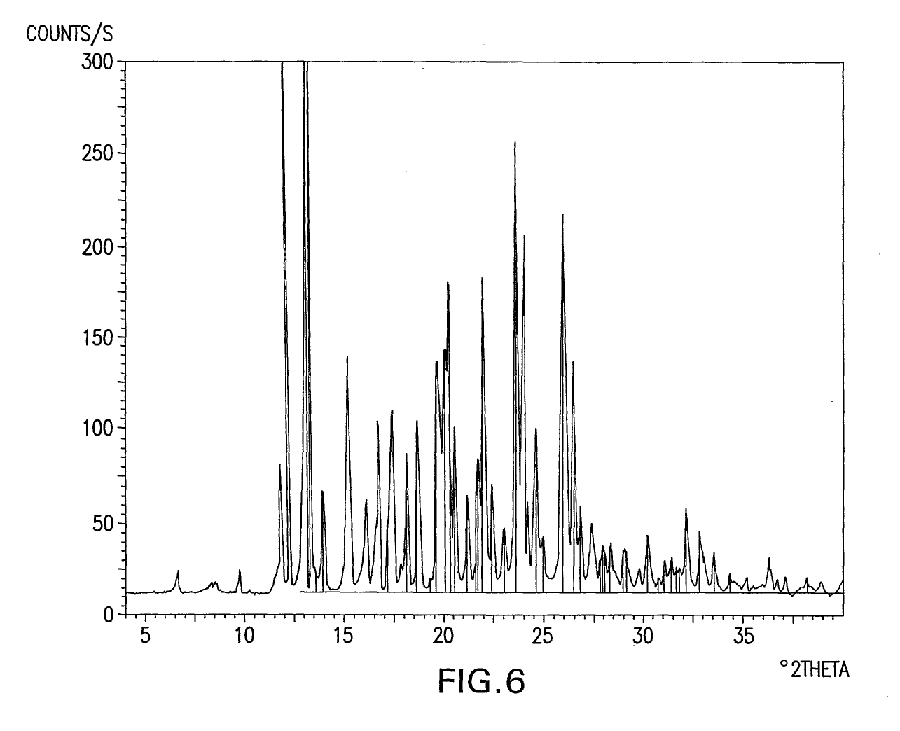

FIG. 7 is the X-ray powder diffraction pattern for 2-butyl-4-chloro-1-[(2′-(1-H-tetrazol-5-yl)biphenyl-4-yl)methyl]-imidazole-5-carboxylic acid monohydrate Form VII.

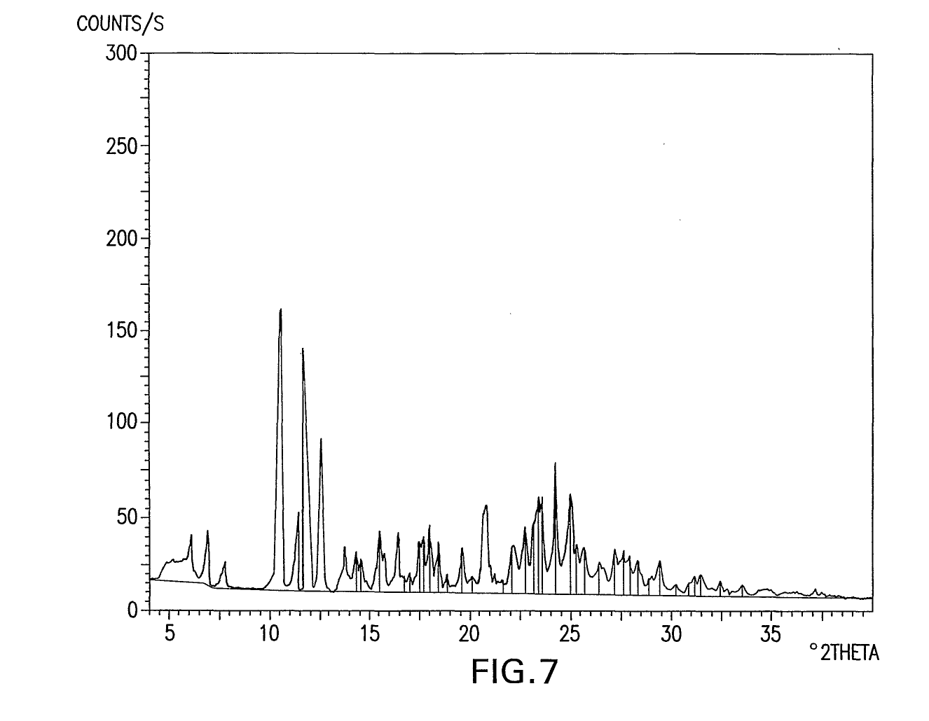

FIG. 8 is the X-ray powder diffraction pattern for 2-butyl-4-chloro-1-[(2′-(1-H-tetrazol-5-yl)biphenyl-4-yl)methyl]-imidazole-5-carboxylic acid monohydrate Form VIII.

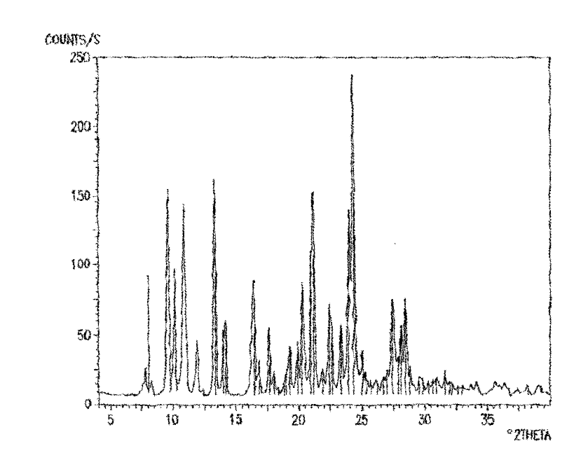

### DETAILED DESCRIPTION OF THE INVENTION AND PREFERRED EMBODIMENTS

The compounds of the present invention are angiotensin II receptor antagonist polymorphic forms of the compound 2-butyl-4-chloro-1-[(2′-(1-H-tetrazol-5-yl)biphenyl-4-yl)methyl]-imidazole-5-carboxylic acid, which has the structure

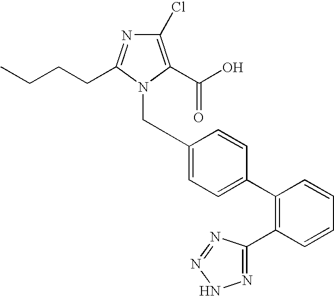

In one embodiment of the invention, the compounds are crystalline forms of 2-butyl-4-chloro-1-[(2′-(1-H-tetrazol-5-yl)biphenyl-4-yl)methyl]-imidazole-5-carboxylic acid having X-ray powder diffraction pattern d-spacing readings selected from the group of readings consisting of
a) about 12.9, about 4.8, about 4.0, about 3.79, about 3.77, about 3.7, and about 3.4 Å,
b) about 8.6, about 6.5, about 5.7, about 3.9, and about 3.7 Å,
c) about 18.8, about 9.5, about 9.3, about 6.2, and about 4.2 Å,
d) about 8.6, about 5.0, about 3.7, and about 3.6 Å,
e) about 6.44, about 6.39, about 6.3, and about 4.2 Å,
f) about 7.4, about 6.8, and about 6.7 Å,
g) about 15.9 Å, and
h) about 9.1, about 8.1, about 6.6, about 4.2, about 3.7 and about 3.7 Å.

In a preferred embodiment of the invention, the compounds are crystalline forms of 2-butyl-4-chloro-1-[(2′-(1-H-tetrazol-5-yl)biphenyl-4-yl)methyl]-imidazole-5-carboxylic acid having X-ray powder diffraction pattern d-spacing readings selected from the group of readings consisting of
a) about 12.96, about 4.75, about 3.97, about 3.79, about 3.77, about 3.71, and about 3.44 Å,
b) about 8.55, about 6.54, about 5.68, about 3.90, and about 3.71 Å,
c) about 18.78, about 9.49, about 9.34, about 6.22, and about 4.20 Å,
d) about 8.57, about 5.01, about 3.66, and about 3.63 Å,
e) about 6.44, about 6.39, about 6.34, and about 4.20 Å,
f) about 7.38, about 6.75, and about 6.69 Å,
g) about 15.91 Å, and
h) about 9.13, about 8.09, about 6.61, about 4.18, about 3.70 and about 3.65 Å.

In a more preferred embodiment of the invention, the compounds are crystalline forms of 2-butyl-4-chloro-1-[(2′-(1-H-tetrazol-5-yl)biphenyl-4-yl)methyl]-imidazole-5-carboxylic acid having X-ray powder diffraction pattern d-spacing readings selected from the group of readings consisting of
a) 12.96, 8.32, 8.13, 7.06, 5.18, 4.75, 4.64, 4.45, 4.41, 4.33, 4.19, 3.97, 3.86, 3.79, 3.77, 3.71, 3.59, 3.44, 3.15, and 2.92 Å,
b) 10.09, 8.55, 7.42, 7.26, 6.54, 6.43, 5.68, 4.39, 4.26, 4.17, 4.12, 4.10, 4.02, 3.90, 3.85, 3.71, 3.67, 3.63, 3.59, 3.44, 3.37, 3.35, 3.12, and 3.02 Å,
c) 18.78, 9.49, 9.34, 6.22, 6.18, 4.85, 4.67, 4.46, 4.20, 3.97, 3.68, 3.66, 3.63, and 3.50 Å,
d) 10.52, 8.57, 7.46, 6.60, 5.45, 5.37, 5.01, 4.91, 4.65, 3.80, 3.66, 3.63, 3.29, 3.23, 3.22, 3.19, 3.02 Å,
e) 7.34, 6.90, 6.44, 6.39, 6.34, 5.69, 5.64, 4.54, 4.26, 4.24, 4.20, 3.91, 3.90, 3.77, and 3.59 Å,
f) 7.38, 7.32, 6.75, 6.69, 4.38, 4.03, 3.76, 3.70, and 3.42 Å,
g) 15.91, 9.99, 8.37, and 7.59 Å, and
h) 10.99, 9.13, 8.69, 8.09, 6.61, 6.32, 6.24, 5.39, 4.37, 4.23, 4.18, 3.96, 3.93, 3.80, 3.70, 3.65, 3.24, 3.17 and 3.13 Å.

Exemplary angiotensin II receptor antagonist polymorphic forms of these embodiments are crystalline forms of 2-butyl-4-chloro-1-[(2′-(1-H-tetrazol-5-yl)biphenyl-4-yl)methyl]-imidazole-5-carboxylic acid selected from the group consisting of 2-butyl-4-chloro-1-[(2′-(1-H-tetrazol-5-yl)biphenyl-4-yl)methyl]-imidazole-5-carboxylic acid hydrochloride Form I, 2-butyl-4-chloro-1-[(2′-(1-H-tetrazol-5-yl)biphenyl-4-yl)methyl]-imidazole-5-carboxylic acid hydrochloride Form II, 2-butyl-4-chloro-1-[(2′-(1-H-tetrazol-5-yl)biphenyl-4-yl)methyl]-imidazole-5-carboxylic acid hydrochloride Form III, and 2-butyl-4-chloro-1-[(2′-(1-H-tetrazol-5-yl)biphenyl-4-yl)methyl]-imidazole-5-carboxylic acid monohydrate Form IV, 2-butyl-4-chloro-1-[(2′-(1-H-tetrazol-5-yl)biphenyl-4-yl)methyl]-imidazole-5-carboxylic acid monohydrate Form V, 2-butyl-4-chloro-1-[(2′-(1-H-tetrazol-5-yl)biphenyl-4-yl)methyl]-imidazole-5-carboxylic acid monohydrate Form VI, 2-butyl-4-chloro-1-[(2′-(1-H-tetrazol-5-yl)biphenyl-4-yl)methyl]-imidazole-5-carboxylic acid monohydrate Form VII, and 2-butyl-4-chloro-1-[(2′-(1-H-tetrazol-5-yl)biphenyl-4-yl)methyl]-imidazole-5-carboxylic acid monohydrate Form VIII. Procedures for preparing these forms, and diffractograms associated with these forms, are provided in the present description.

### Angiotensin II Receptor Antagonists—Therapeutic Uses—Method of Using

The angiotensin II receptor antagonist polymorphic forms of the invention are useful for the treatment and/or prophylaxis of diseases which are related to hypertension, congestive heart failure, pulmonary hypertension, renal insufficiency, renal ischemia, renal failure, renal fibrosis, cardiac insufficiency, cardiac hypertrophy, cardiac fibrosis, myocardial ischemia, cardiomyopathy, glomerulonephritis, renal colic, complications resulting from diabetes such as nephropathy, vasculopathy and neuropathy, glaucoma, elevated intra-ocular pressure, atherosclerosis, restenosis post angioplasty, complications following vascular or cardiac surgery, erectile dysfunction, hyperaldosteronism, lung fibrosis, scleroderma, anxiety, cognitive disorders, complications of treatments with immunosuppressive agents, and other diseases known to be related to the renin-angiotensin system.

The angiotensin II receptor antagonist polymorphic forms of the invention are especially useful for the treatment and/or prophylaxis of diseases which are related to hypertension, congestive heart failure, pulmonary hypertension, renal insufficiency, renal ischemia, renal failure, renal fibrosis, cardiac insufficiency, cardiac hypertrophy, cardiac fibrosis, myocardial ischemia, cardiomyopathy, complications resulting from diabetes such as nephropathy, vasculopathy and neuropathy.

In one embodiment, the invention relates to a method for the treatment and/or prophylaxis of diseases, which are associated with a dysregulation of the renin-angiotensin system, in particular to a method for the treatment or prophylaxis of the above-mentioned diseases, said methods comprising administering to a patient a pharmaceutically active amount of an angiotensin II receptor antagonist of the invention.

The invention also relates to the use of angiotensin II receptor antagonist polymorphic forms of the invention for the preparation of a medicament for the treatment and/or prophylaxis of the above-mentioned diseases.

The above-mentioned angiotensin II receptor antagonist polymorphic forms of the invention are also of use in combination with other pharmacologically active compounds comprising angiotensin converting enzyme inhibitors (e.g, alacepril, benazepril, captopril, ceronapril, cilazapril, delapril, enalapril, enalaprilat, fosinopril, imidapril, lisinopril, moveltipril, perindopril, quinapril, ramipril, spirapril, temocapril, or trandolapril), neutral endopeptidase inhibitors (e.g., thiorphan and phosphoramidon), aldosterone antagonists, renin inhibitors (e.g. urea derivatives of di- and tri-peptides (See U.S. Pat. No. 5,116,835), amino acids and derivatives (U.S. Pat. Nos. 5,095,119 and 5,104,869), amino acid chains linked by non-peptidic bonds (U.S. Pat. No. 5,114,937), di- and tri-peptide derivatives (U.S. Pat. No. 5,106,835), peptidyl amino diols (U.S. Pat. Nos. 5,063,208 and 4,845,079) and peptidyl beta-aminoacyl aminodiol carbamates (U.S. Pat. No. 5,089,471); also, a variety of other peptide analogs as disclosed in the following U.S. Pat. Nos. 5,071,837; 5,064,965; 5,063,207; 5,036,054; 5,036,053; 5,034,512 and 4,894,437, and small molecule renin inhibitors (including diol sulfonamides and sulfinyls (U.S. Pat. No. 5,098,924), N-morpholino derivatives (U.S. Pat. No. 5,055,466), N-heterocyclic alcohols (U.S. Pat. No. 4,885,292) and pyrolimidazolones (U.S. Pat. No. 5,075,451); also, pepstatin derivatives (U.S. Pat. No. 4,980,283) and fluoro- and chloro-derivatives of statone-containing peptides (U.S. Pat. No. 5,066,643), enalkrein, RO 42-5892, A 65317, CP 80794, ES 1005, ES 8891, SQ 34017, aliskiren (2(S),4(S),5(S),7(S)—N-(2-carbamoyl-2-methylpropyl)-5-amino-4-hydroxy-2,7-diisopropyl-8-[4-methoxy-3-(3-methoxypropoxy)phenyl]-octanamid hemifumarate) SPP600, SPP630 and SPP635), endothelin receptors antagonists, vasodilators, calcium channel blockers (e.g., amlodipine, nifedipine, veraparmil, diltiazem, gallopamil, niludipine, nimodipins, nicardipine), potassium channel activators (e.g., nicorandil, pinacidil, cromakalim, minoxidil, aprilkalim, loprazolam), diuretics (e.g., hydrochlorothiazide), sympatholitics, beta-adrenergic blocking drugs (e.g., propranolol, atenolol, bisoprolol, carvedilol, metoprolol, or metoprolol tartate), alpha adrenergic blocking drugs (e.g., doxazocin, prazocin or alpha methyldopa) central alpha adrenergic agonists, peripheral vasodilators (e.g. hydralazine), lipid lowering agents (e.g., simvastatin, lovastatin, ezetamibe, atorvastatin, pravastatin), metabolic altering agents including insulin sensitizing agents and related compounds (e.g., muraglitazar, glipizide, metformin, rosiglitazone)) or with other drugs beneficial for the prevention or the treatment of the above-mentioned diseases including nitroprusside and diazoxide.

The dosage regimen utilizing the angiotensin II receptor antagonist polymorphic forms is selected in accordance with a variety of factors including type, species, age, weight, sex and medical condition of the patient; the severity of the condition to be treated; the route of administration; the renal and hepatic function of the patient; and the particular compound or salt thereof employed. An ordinarily skilled physician or veterinarian can readily determine and prescribe the effective amount of the drug required to prevent, counter, or arrest the progress of the condition.

Oral dosages of the angiotensin II receptor antagonist polymorphic forms, when used for the indicated effects, will range between about 0.0125 mg per kg of body weight per day (mg/kg/day) to about 7.5 mg/kg/day, preferably 0.0125 mg/kg/day to 3.75 mg/kg/day, and more preferably 0.3125 mg/kg/day to 1.875 mg/kg/day. For example, an 80 kg patient would receive between about 1 mg/day and 600 mg/day, preferably 1 mg/day to 300 mg/day, and more preferably 25 mg/day to 150 mg/day. A suitably prepared medicament for once a day administration would thus contain between 1 mg and 600 mg, preferably between 1 mg and 300 mg, and more preferably between 25 mg and 300 mg, e.g., 25 mg, 50 mg, 100 mg, 150, 200, 250 and 300 mg. Advantageously, the angiotensin H receptor antagonists may be administered in divided doses of two, three, or four times daily. For administration twice a day, a suitably prepared medicament would contain between 0.5 mg and 300 mg, preferably between 0.5 mg and 150 mg, more preferably between 12.5 mg and 150 mg, e.g., 12.5 mg, 25 mg, 50 mg, 75 mg, 100 mg, 125 mg and 150 mg.

Formulation amounts in the composition of the invention are represented by the weight amount of the specified ingredient added to the formulation, e.g. an amount of 2-butyl-4-chloro-1-[(2′-(1-H-tetrazol-5-yl)biphenyl-4-yl)methyl]-imidazole-5-carboxylic acid hydrochloride added to a formulation includes both the free form and salt components of the 2-butyl-4-chloro-1-[(2′-(1-H-tetrazol-5-yl)biphenyl-4-yl)methyl]-imidazole-5-carboxylic acid hydrochloride. In the case of 2-butyl-4-chloro-1-[(2′-(1-H-tetrazol-5-yl)biphenyl-4-yl)methyl]-imidazole-5-carboxylic acid hydrochloride, for example, a formulation containing 108.5 mg of 2-butyl-4-chloro-1-[(2′-(1-H-tetrazol-5-yl)biphenyl-4-yl)methyl]-imidazole-5-carboxylic acid hydrochloride provides 100 mg of the free form component and 8.5 mg of the hydrochloride component.

The angiotensin II receptor antagonist polymorphic forms of the invention can be administered in such oral forms as tablets, capsules and granules. The angiotensin II receptor antagonists are typically administered as active ingredients in admixture with suitable pharmaceutical binders as described below. % w/w expresses the weight percent of the indicated composition constituent compared to the total composition.

One embodiment of a suitable pharmaceutical composition for administering angiotensin II receptor antagonist polymorphic forms of the present invention is a pharmaceutical particle matrix composition comprising

a) an amount between about 1 and 75% w/w of a polymorphic form of 2-butyl-4-chloro-1-[(2′-(1-H-tetrazol-5-yl)biphenyl-4-yl)methyl]-imidazole-5-carboxylic acid having X-ray powder diffraction pattern d-spacing readings selected from the group of readings consisting of

i) about 12.9, about 4.8, about 4.0, about 3.79, about 3.77, about 3.7, and about 3.4 Å,

ii) about 8.6, about 6.5, about 5.7, about 3.9, and about 3.7 Å,

iii) about 18.8, about 9.5, about 9.3, about 6.2, and about 4.2 Å,

iv) about 8.6, about 5.0, about 3.7, and about 3.6 Å,

v) about 6.44, about 6.39, about 6.3, and about 4.2 Å,

vi) about 7.4, about 6.8, and about 6.7 Å,

vii) about 15.9 Å, and

viii) about 9.1, about 8.1, about 6.6, about 4.2, about 3.7 and about 3.7 Å;

b) an amount between about 2.5 and 90% w/w of at least one water swellable polymer;
c) an amount between about 2.5 and 90% w/w of at least one filler; and
d) an amount between about 0.5 and 10% w/w of at least one lubricant,
wherein the particle size of the particle is between about 50 and 1200 μm.

The particle matrix composition optionally includes an amount between 2.5 and 15% w/w of a solubilizing agent.

Preferably, the angiotensin II receptor antagonist polymorphic form is selected from the group consisting of 2-butyl-4-chloro-1-[(2′-(1-H-tetrazol-5-yl)biphenyl-4-yl)methyl]-imidazole-5-carboxylic acid hydrochloride Form I, 2-butyl-4-chloro-1-[(2′-(1-H-tetrazol-5-yl)biphenyl-4-yl)methyl]-imidazole-5-carboxylic acid hydrochloride Form II, 2-butyl-4-chloro-1-[(2′-(1-H-tetrazol-5-yl)biphenyl-4-yl)methyl]-imidazole-5-carboxylic acid hydrochloride Form III, and 2-butyl-4-chloro-1-[(2′-(1-H-tetrazol-5-yl)biphenyl-4-yl)methyl]-imidazole-5-carboxylic acid monohydrate Form IV, 2-butyl-4-chloro-1-[(2′-(1-H-tetrazol-5-yl)biphenyl-4-yl)methyl]-imidazole-5-carboxylic acid monohydrate Form V, 2 butyl-4-chloro-1-[(2′-(1-H-tetrazol-5-yl)biphenyl-4-yl)methyl]-imidazole-5-carboxylic acid monohydrate Form VI, 2-butyl-4-chloro-1-[(2′-(1-H-tetrazol-5-yl)biphenyl-4-yl)methyl]-imidazole-5-carboxylic acid monohydrate Form VII, and 2-butyl-4-chloro-1-[(2′-(1-H-tetrazol-5-yl)biphenyl-4-yl)methyl]-imidazole-5-carboxylic acid monohydrate Form VII.

In this embodiment, one or more water swellable polymer is preferably selected from the group consisting of polyethylene oxide and hydroxypropylmethyl cellulose, the filler is preferably a mixture of dicalcium phosphate and microcrystalline cellulose, and the lubricant is preferably magnesium stearate. These particles may be administered to the patient as the sole active ingredient formulation, or mixed with immediate release granules of the angiotensin II receptor antagonists in proportions ranging from 1:10 to 10:1 appropriate for achieving the desired overall release rate.

Another embodiment of a suitable pharmaceutical composition for administering angiotensin II receptor antagonist polymorphic forms of the present invention is an erodible matrix composition comprising

a) an amount between about 1 and 90% w/w of a polymorphic form of 2-butyl-4-chloro-1-[(2′-(1-H-tetrazol-5-yl)biphenyl-4-yl)methyl]-imidazole-5-carboxylic acid having X-ray powder diffraction pattern d-spacing readings selected from the group of readings consisting of

i) about 12.9, about 4.8, about 4.0, about 3.79, about 3.77, about 3.7, and about 3.4 Å,

ii) about 8.6, about 6.5, about 5.7, about 3.9, and about 3.7 Å,

iii) about 18.8, about 9.5, about 9.3, about 6.2, and about 4.2 Å,

iv) about 8.6, about 5.0, about 3.7, and about 3.6 Å,

v) about 6.44, about 6.39, about 6.3, and about 4.2 Å,

vi) about 7.4, about 6.8, and about 6.7 Å,

vii) about 15.9 Å, and

viii) about 9.1, about 8.1, about 6.6, about 4.2, about 3.7 and about 3.7 Å;

b) an amount between about 2.5 and 25% w/w of at least one water swellable polymer;
c) an amount between about 2.5 and 15% w/w of at least one solubilizing agent; and
d) an amount between about 2.5 and 90% w/w of at least one filler.

The erodible matrix composition optionally includes up to 15% w/w of a water soluble filler.

Preferably, the angiotensin II receptor antagonist polymorphic form is selected from the group consisting of 2-butyl-4-chloro-1-[(2′-(1-H-tetrazol-5-yl)biphenyl-4-yl)methyl]-imidazole-5-carboxylic acid hydrochloride Form I, 2-butyl-4-chloro-1-[(2′-(1-H-tetrazol-5-yl)biphenyl-4-yl)methyl]-imidazole-5-carboxylic acid hydrochloride Form II, 2-butyl-4-chloro-1-[(2′-(1-H-tetrazol-5-yl)biphenyl-4-yl)methyl]-imidazole-5-carboxylic acid hydrochloride Form III, and 2-butyl-4-chloro-1-[(2′-(1-H-tetrazol-5-yl)biphenyl-4-yl)methyl]-imidazole-5-carboxylic acid monohydrate Form IV, 2-butyl-4-chloro-1-[(2′-(1-H-tetrazol-5-yl)biphenyl-4-yl)methyl]-imidazole-5-carboxylic acid monohydrate Form V, 2-butyl-4-chloro-1-[(2′-(1-H-tetrazol-5-yl)biphenyl-4-yl)methyl]-imidazole-5-carboxylic acid monohydrate Form VI, 2-butyl-4-chloro-1-[(2′-(1-H-tetrazol-5-yl)biphenyl-4-yl)methyl]-imidazole-5-carboxylic acid monohydrate Form VII, and 2-butyl-4-chloro-1-[(2′-(1-H-tetrazol-5-yl)biphenyl-4-yl)methyl]-imidazole-5-carboxylic acid monohydrate Form VII.

In this embodiment, one or more water swellable polymer is selected from the group consisting of polyethylene oxide and hydroxypropylmethyl cellulose, the bulking agent is a mixture of dicalcium phosphate, microcrystalline cellulose and lactose, and the solubilizing agent is selected from the group of block copolymers of ethylene oxide and propylene oxide (e.g., Poloxamers).

Another embodiment of a suitable pharmaceutical composition for administering angiotensin II receptor antagonist polymorphic forms of the present invention is a granule and a coating material coating the granule, wherein

a) the granule comprises

1) an amount between about 5 and 50% w/w of a polymorphic form of 2-butyl-4-chloro-1-[(2′-(1-H-tetrazol-5-yl)biphenyl-4-yl)methyl]-imidazole-5-carboxylic acid having X-ray powder diffraction pattern d-spacing readings selected from the group of readings consisting of

i) about 12.9, about 4.8, about 4.0, about 3.79, about 3.77, about 3.7, and about 3.4 Å,

ii) about 8.6, about 6.5, about 5.7, about 3.9, and about 3.7 Å,

iii) about 18.8, about 9.5, about 9.3, about 6.2, and about 4.2 Å,

iv) about 8.6, about 5.0, about 3.7, and about 3.6 Å,

v) about 6.44, about 6.39, about 6.3, and about 4.2 Å,

vi) about 7.4, about 6.8, and about 6.7 Å, vii) about 15.9 Å, and

viii) about 9.1, about 8.1, about 6.6, about 4.2, about 3.7 and about 3.7 Å;

2) an amount between about 5 and 50% w/w of at least one neutralizing agent;

3) an amount between about 1 and 25% w/w of at least one binder; and

4) an amount between about 5 and 75% w/w of at least one filler,

wherein the particle size of the granule is between about 100 and 1200 μm; and
b) the coating material comprises
1) a polymer deposited in the form of an aqueous dispersion or organic solution with subsequent evaporation of the dispersion solvents, wherein the amount of polymer in the coating material is such that the amount of polymer deposited on the granule is between about 5 and 40% w/w of the granule;
2) a plasticizer in the amount of up to 40% of the polymer weight; and
3) an anti-tacking agent in the amount of up to 10% of the polymer weight.

Preferably, the angiotensin II receptor antagonist polymorphic form is selected from the group consisting of 2-butyl-4-chloro-1-[(2′-(1-H-tetrazol-5-yl)biphenyl-4-yl)methyl]-imidazole-5-carboxylic acid hydrochloride Form I, 2-butyl-4-chloro-1-[(2′-(1-H-tetrazol-5-yl)biphenyl-4-yl)methyl]-imidazole-5-carboxylic acid hydrochloride Form II, 2-butyl-4-chloro-1-[(2′-(1-H-tetrazol-5-yl)biphenyl-4-yl)methyl]-imidazole-5-carboxylic acid hydrochloride Form II, and 2-butyl-4-chloro-1-[(2′-(1-H-tetrazol-5-yl)biphenyl-4-yl)methyl]-imidazole-5-carboxylic acid monohydrate Form IV, 2-butyl-4-chloro-1-[(2′-(1-H-tetrazol-5-yl)biphenyl-4-yl)methyl]-imidazole-5-carboxylic acid monohydrate Form V, 2-butyl-4-chloro-1-[(2′-(1-H-tetrazol-5-yl)biphenyl-4-yl)methyl]-imidazole-5-carboxylic acid monohydrate Form VI, 2-butyl-4-chloro-1-[(2′-(1-H-tetrazol-5-yl)biphenyl-4-yl)methyl]-imidazole-5-carboxylic acid monohydrate Form VII, and 2-butyl-4-chloro-1-[(2′-(1-H-tetrazol-5-yl)biphenyl-4-yl)methyl]-imidazole-5-carboxylic acid monohydrate Form VIII.

The coating material comprises between about 5 and about 60% of the granule weight, i.e., a composition comprising a 100 mg granule further comprises between about 5 mg and about 60 mg of coating material.

The granule optionally includes an amount between about 2.5 and 15% w/w of a solubilizing agent and also optionally includes an amount of up to 15% w/w of a water soluble filler.

In this embodiment, the neutralizing agent is dibasic sodium phosphate heptahydrate, the filler is microcrystalline cellulose, the binder is hydroxypropyl cellulose, and the aqueous dispersion comprises ethyl cellulose, triethyl citrate and kaolin. These coated granules may be administered to the patient as the sole active ingredient formulation, or mixed with immediate release granules of the angiotensin HI receptor antagonists in proportions ranging from 1:10 to 10:1 appropriate for achieving the desired overall release rate.

Suitable fillers used in these dosage forms include microcrystalline cellulose, silicified microcrystalline cellulose, dicalcium phosphate, lactose, mannitol, and starch, preferably microcrystalline cellulose, dicalcium phosphate, lactose or mixtures thereof.

Suitable binders include hydroxypropyl cellulose, hydroxypropyl methyl cellulose, starch, gelatin, natural sugars such as glucose or beta-lactose, corn-sweeteners, natural and synthetic gums such as acacia, tragacanth or sodium alginate, carboxymethylcellulose, and polyvinyl pyrrolidone.

Lubricants used in these dosage forms include sodium oleate, sodium stearate, magnesium stearate, sodium benzoate, sodium acetate, sodium chloride, sodium stearyl fumarate, stearic acid and the like, preferably magnesium stearate.

Water swellable polymers are polymers capable of swelling upon hydration. The term “swellable” implies that the polymer is in a non-hydrated state, while the term “swelling” implies that the polymer is in a hydrated state. These polymers generally have high molecular weights ranging from 25,000 to 10,000,000 with correspondingly high viscosities in hydrated state. Illustrative examples of swellable polymers are cellulosic polymers like hydroxypropylmethyl cellulose, hydroxypropyl cellulose, hydroxyethyl cellulose, methyl cellulose, sodium carboxymethylcellulose, calcium carboxymethyl cellulose and non-cellulosics like polyethylene oxide, polyvinyl alcohol, guargum, acacia, carageenan, tragacanth, alginates, pectin, and xanthan gum. Also illustrative are “AQUAKEEP J-550”, “AQUAKEEP J-400”, which are trade names for sodium acrylate polymer produced by Seitetsu Kagaku Co., Ltd., Hyogo, Japan. The “AQUAKEEP” polymers are generically described in U.S. Pat. No. 4,340,706. Also illustrative of this type of polymer are the carboxypolymethylenes prepared from acrylic acid cross-linked with allyl ethers of sucrose or pentaerytlritol and sold under the trade names “CARBOPOL 934P” and “CARBOPOL 974P” which are trade names for two carbamer type polymers produced by B.F. Goodrich Chemical Company, Cleveland, Ohio. Carbamer polymers are generically described in U.S. Pat. No. 2,909,462 and in the National Formulary XVII at p. 1911, CAS Registry Number 9003-01-4. All of the forgoing references are hereby incorporated by reference. In the dry state, “CARBOPOL 974P” and “CARBOPOL 934P” particles range in size from 2 to 7 microns. When these dry particles are hydrated, microscopic gel particles in the range of 20 microns are produced. When “AQUAKEEP J-550” or “AQUAKEEP J-400” dry particles are hydrated, microscopic gel particles diameter can range in size from 100 to 1000 microns. Preferably, the water swellable polymers are polyethylene oxide and hydroxypropylmethyl cellulose.

Suitable solubilizing agents include poloxamers and fatty acid macrogolglycerides. Poloxamers are block copolymers of ethylene oxide and propylene oxide. Suitable poloxamers include, for example, those having an average molecular weight in a range of from about 1000 to about 20,000 and an oxyethylene content of from about 40 to about 90 wt. % Representative poloxamers suitable for use in the present invention include poloxamer 188, poloxamer 237, poloxamer 338, and poloxamer 407. A suitable fatty acid macrogolglyceride is stearoyl macrogolglyceride, such as GELUCIRE® 50/13 (available from Gattefosse, Paramus, N.J.) which is a mixture of mono-, di- and triglycerides and mono- and di-fatty acid esters of polyethylene glycol with a melting range of 46.0 to 51.0° C. and an HLB value of 13.

Suitable coating compositions include aqueous dispersion or organic solution of insoluble polymers such as ethyl cellulose, cellulose acetate, cellulose acetate butyrate and acrylate copolymers commercially known as Eudragit®. Plasticizers include triethyl citrate, dibutyl sebacate, dibutyl phthalate, triacetin and castor oil.

Antitacking agents include talc, kaolin, colloidal silica or mixtures thereof.

By “drug delivery device” is meant, a dosage form that provides a convenient means of delivering a pharmaceutically active ingredient or drug to a subject in need thereof. The subject can be a human or any other animal in need of such pharmaceutically active ingredient. The device is designed to be useful for the delivery of a pharmaceutically active ingredient by any pharmaceutically accepted means such as by swallowing, retaining it within the mouth until the beneficial agent has been dispensed, placing it within the bucal cavity, or the like.

By “controlled” is meant that the rate of release of the pharmaceutically active ingredient from the device to the environment of use is not immediate, but rather, follows a predetermined pattern. Thus, relatively constant or predictably varying amounts of the beneficial agent can be dispensed over a specified period of time.

“Polymorphs” are different crystalline forms of the same substance in which the molecules have different arrangements and/or different conformations of the molecules. They display different physical properties, including, for example, one or more of the following: those due to packing, various thermodynamic, spectroscopic, interfacial and mechanical properties.

In the examples below, “Active Ingredient” is any one of the compounds selected from the group consisting of 2-butyl-4-chloro-1-[(2′-(1-H-tetrazol-5-yl)biphenyl-4-yl)methyl]-imidazole-5-carboxylic acid hydrochloride Form I, 2-butyl-4-chloro-1-[(2′-(1-H-tetrazol-5-yl)biphenyl-4-yl)methyl]-imidazole-5-carboxylic acid hydrochloride Form II, 2-butyl-4-chloro-1-[(2′-(1-H-tetrazol-5-yl)biphenyl-4-yl)methyl]-imidazole-5-carboxylic acid hydrochloride Form III, and 2-butyl-4-chloro-1-[(2′-(1-H-tetrazol-5-yl)biphenyl-4-yl)methyl]-imidazole-5-carboxylic acid monohydrate Form IV, 2-butyl-4-chloro-1-[(2′-(1-H-tetrazol-5-yl)biphenyl-4-yl)methyl]-imidazole-5-carboxylic acid monohydrate Form V, 2-butyl-4-chloro-1-[(2′-(1-H-tetrazol-5-yl)biphenyl-4-yl)methyl]-imidazole-5-carboxylic acid monohydrate Form VI, 2-butyl-4-chloro-1-[(2′-(1-H-tetrazol-5-yl)biphenyl-4-yl)methyl]-imidazole-5-carboxylic acid monohydrate Form VU, and 2-butyl-4-chloro-1-[(2′-(1-H-tetrazol-5-yl)biphenyl-4-yl)methyl]-imidazole-5-carboxylic acid monohydrate Form VIII.

2-Butyl-4-chloro-1-[(2′-(1-H-tetrazol-5-yl)biphenyl-4-yl)methyl]-imidazole-5-carboxylic acid is the active metabolite of 2-butyl-4-chloro-1-[p-(o-1H-tetrazol-5-ylphenyl)-benzyl]imidazole-5-methanol which is available as a monopotassium salt (also known as losartan potassium salt). Losartan potassium salt is available commercially as the active ingredient in COZAAR® (Merck & Co., Inc. (Whitehouse Station, N.J.)). The preparation of losartan potassium salt is described in U.S. Pat. Nos. 5,136,069, 5,130,439, and 5,310,928. Tetrazolylphenylboronic acid intermediates useful in the synthesis of losartan potassium salt are described in U.S. Pat. No. 5,206,374. Additional patents which describe procedures useful for making losartan include U.S. Pat. Nos. 4,820,843, 4,870,186, 4,874,867, 5,039,814, and 5,859,258.

### Example I

### Preparation of 2-butyl-4-chloro-1-[(2′-(1-H-tetrazol-5-yl)biphenyl-4-yl)methyl]-imidazole-5-carboxylic Acid Hydrochloride Form I

To a 22 liter flask equipped with a thermocouple, nitrogen inlet, and mechanical stirrer was added 14 liters of water. To the solution was added 1197 grams (86%) potassium hydroxide pellets and 700 grams losartan potassium (2-butyl-4-chloro-1-[p-(o-1H-tetrazol-5-ylphenyl)-benzyl]imidazole-5-methanol), rinsed in with 500 ml water. The resulting solution was cooled to 2° C. in an isopropanol bath cooled with a chiller unit. Cooling took 2 hours. During this time, a white slurry formed. 775 grams sodium periodate, followed by 16.9 grams ruthenium (III) chloride hydrate, was added to the slurry, rinsed in with 100 ml water. The temperature rose to 6-6.5° C. The resulting slurry was aged at 4-6° C. After aging 19 hours, the reaction was complete. After filtering off the solids and washing with 700 ml water, add 130 ml isopropyl alcohol to the filtrate and age 2.5 hours at 18-23° C. Filter the mixture through a pad of Solka Floc and wash with 600 ml water. The filtrate was acidified with 1.68 L phosphoric acid while maintaining temperature below 31° C. After aging 30 min, the slurry was filtered and washed with 2×4 L water. The dried solids were subsequently decolorized and concentrated resulting in a white slurry that was filtered and washed with 500 ml of cold methanol and air dried.

460 grams of free acid was dissolved in 5 liters of acetic acid, glacial. The solution was filtered through a glass frit. The filtrate was added to 6 liters of 1.1 N HCl in acetic acid, glacial over 1 hour. Crystallization began just prior to the end of filtrate addition, and the resulting material was aged for 1 hour. An ice and water bath was added to cool the batch, and the batch was aged 1 hour. 12.5 liters of isopropyl acetate was added over 1 hour, and the resulting material was aged at 3-4° C. for 1 hour. Resulting material was filtered and washed with 1 liter isopropyl acetate displacement wash, 2×1 liter isopropyl acetate slurry wash, and 2.5 liter isopropyl acetate displacement wash. Resulting material was air dried 45 minutes, then dried by pulling nitrogen through filter cake overnight followed by drying at 35° C. in a vacuum oven until constant weight. The resulting hydrochloric acid salt was 2-butyl-4-chloro-1-[(2′-(1-H-tetrazol-5-yl)biphenyl-4-yl)methyl]-imidazole-5-carboxylic acid hydrochloride Form I. The solid was then analyzed by X-ray Powder Diffraction. The diffractogram for 2-butyl-4-chloro-1-[(2′-(1-H-tetrazol-5-yl)biphenyl-4-yl)methyl]-imidazole-5-carboxylic acid hydrochloride Form I is characterized by principal d-spacings of 12.96, 4.75, 3.97, 3.79, 3.77, 3.71, and 3.44 Å, more specifically 12.96, 8.32, 8.13, 7.06, 5.18, 4.75, 4.64, 4.45, 4.41, 4.33, 4.19, 3.97, 3.86, 3.79, 3.77, 3.71, 3.59, 3.44, 3.15, and 2.92 Å.

### Example 2

### Preparation of 2-butyl-4-chloro-1-[(2′-(1-H-tetrazol-5-yl)biphenyl-4-yl)methyl]-imidazole-5-carboxylic Acid Hydrochloride Form II

50 mgs of Form I formed in Example 1 was slurried in approximately 0.5 mLs of methanol in a centrifuge tube. The sample was allowed to rotate on a rotator for approximately 72 hours. After 72 hours, the sample was centrifuged and the supernatant was removed. The residual solid was allowed to dry overnight and vacuum-dried (50° C.) for 24 hours. The solid was then analyzed by X-ray Powder Diffraction. The diffractogram for 2-butyl-4-chloro-1-[(2′-(1-H-tetrazol-5-yl)biphenyl-4-yl)methyl]-imidazole-5-carboxylic acid hydrochloride Form II is characterized by principal d-spacings of 8.55, 6.54, 5.68, 3.90, 3.71 Å, more specifically 10.09, 8.55, 7.42, 7.26, 6.54, 6.43, 5.68, 4.39, 4.26, 4.17, 4.12, 4.10, 4.02, 3.90, 3.85, 3.71, 3.67, 3.63, 3.59, 3.44, 3.37, 3.35, 3.12, and 3.02 Å.

### Example 3

### Preparation of 2-butyl-4-chloro-1-[(2′-(1-H-tetrazol-5-yl)biphenyl-4-yl)methyl]-imidazole-5-carboxylic Acid Hydrochloride Form III

50 mgs of Form I formed in Example 1 was slurried in approximately 0.5 mLs of ethanol in a centrifuge tube. The sample was allowed to rotate on a rotator for approximately 72 hours. After 72 hours, the sample was centrifuged and the supernatant was removed. The residual solid was allowed to dry overnight and vacuum-dried (50° C.) for 24 hours. The solid was then analyzed by X-ray Powder Diffraction. The diffractogram for 2-butyl-4-chloro-1-[(2′-(1-H-tetrazol-5-yl)biphenyl-4-yl)methyl]-imidazole-5-carboxylic acid hydrochloride Form III is characterized by principal d-spacings of 18.78, 9.49, 9.34, 6.22, and 4.20 Å, more specifically 18.78, 9.49, 9.34, 6.22, 6.18, 4.85, 4.67, 4.46, 4.20, 3.97, 3.68, 3.66, 3.63, and 3.50 Å.

### Example 4

### Preparation of 2-butyl-4-chloro-1-[(2′-(1-H-tetrazol-5-yl)biphenyl-4-yl)methyl]-imidazole-5-carboxylic Acid Monohydrate Form IV

25 mgs of Form II formed in Example 2 and Form III formed in Example 3 were placed in separate centrifuge tubes. Approximately 25 mgs of Form I formed in Example 1 was added to each centrifuge tube containing Form II and Form III). 0.5 mLs of water was added to each centrifuge tube. The samples were allowed to rotate on a rotator for approximately 72 hours. After 72 hours, the samples were centrifuged and the supernatant was removed. The residual solids were vacuum-dried (40° C.) for 24 hours. The solids were then analyzed by X-ray Powder Diffraction. The diffractogram for 2-butyl-4-chloro-1-[(2′-(1-H-tetrazol-5-yl)biphenyl-4-yl)methyl]-imidazole-5-carboxylic acid monohydrate Form IV is characterized by principal d-spacings of 8.57, 5.01, 3.66, and 3.63 Å, more specifically 10.52, 8.57, 7.46, 6.60, 5.45, 5.37, 5.01, 4.91, 4.65, 3.80, 3.66, 3.63, 3.29, 3.23, 3.22, 3.19, 3.02 Å.

### Example 5

### Alternative Preparation of 2-butyl-4-chloro-1-[(2′-(1-H-tetrazol-5-yl)biphenyl-4-yl)methyl]-imidazole-5-carboxylic Acid Monohydrate Form IV

50 mgs of Form I formed in Example 1 was slurried in approximately 0.5 mLs of water in a centrifuge tube. The sample was allowed to rotate on a rotator for approximately 72 hours. After 72 hours, the sample was centrifuged and the supernatant was removed. The residual solid was allowed to dry overnight and vacuum-dried (50° C.) for 24 hours. The solid was then analyzed by X-ray Powder Diffraction. The diffractogram for 2-butyl-4-chloro-1-[(2′-(1-H-tetrazol-5-yl)biphenyl-4-yl)methyl]-imidazole-5-carboxylic acid monohydrate Form IV is characterized by principal d-spacings of 8.57, 5.01, 3.66, and 3.63 Å, more specifically 10.52, 8.57, 7.46, 6.60, 5.45, 5.37, 5.01, 4.91, 4.65, 3.80, 3.66, 3.63, 3.29, 3.23, 3.22, 3.19, 3.02 Å.

### Example 6

### Preparation of 2-butyl-4-chloro-1-[(2′-(1-H-tetrazol-5-yl)biphenyl-4-yl)methyl]-imidazole-5-carboxylic Acid Monohydrate Form V

50 mgs of Form IV formed in Example 5 was slurried in approximately 0.5 mLs of methanol in a centrifuge tube. The sample was allowed to rotate on a rotator for approximately 72 hours. After 72 hours, the sample was centrifuged and the supernatant was removed. The residual solid was allowed to dry overnight and vacuum-dried (50° C.) for 24 hours. The solid was then analyzed by X-ray Powder Diffraction. The diffractogram for 2-butyl-4-chloro-1-[(2′-(1-H-tetrazol-5-yl)biphenyl-4-yl)methyl]-imidazole-5-carboxylic acid monohydrate Form V is characterized by principal d-spacings of 6.44, 6.39, 6.34, and 4.20 Å, more specifically 7.34, 6.90, 6.44, 6.39, 6.34, 5.69, 5.64, 4.54, 4.26, 4.24, 4.20, 3.91, 3.90, 3.77, and 3.59 Å.

### Example 7

### Preparation of 2-butyl-4-chloro-1-[(2′-(1-H-tetrazol-5-yl)biphenyl-4-yl)methyl]-imidazole-5-carboxylic Acid Monohydrate Form VI

50 mgs of Form IV formed in Example 5 was slurried in approximately 0.5 mLs of ethanol in a centrifuge tube. The sample was allowed to rotate on a rotator for approximately 72 hours. After 72 hours, the sample was centrifuged and the supernatant was removed. The residual solid was allowed to dry overnight and vacuum-dried (50° C.) for 24 hours. The solid was then analyzed by X-ray Powder Diffraction. The diffractogram for 2-butyl-4-chloro-1-[(2′-(1-H-tetrazol-5-yl)biphenyl-4-yl)methyl]-imidazole-5-carboxylic acid monohydrate Form VI is characterized by principal d-spacings of 7.38, 6.75, and 6.69 Å, more specifically 7.38, 7.32, 6.75, 6.69, 4.38, 4.03, 3.76, 3.70, and 3.42 Å.

### Example 8

### Preparation of 2-butyl-4-chloro-1-[(2′-(1-H-tetrazol-5-yl)biphenyl-4-yl)methyl]-imidazole-5-carboxylic Acid Monohydrate Form VII

50 mgs of Form IV formed in Example 5 was slurried in approximately 0.5 mLs of acetonitrile in a centrifuge tube. The sample was allowed to rotate on a rotator for approximately 72 hours. After 72 hours, the sample was centrifuged and the supernatant was removed. The residual solid was allowed to dry overnight and vacuum-dried (50° C.) for 24 hours. The solid was then analyzed by X-ray Powder Diffraction. The diffractogram for 2-butyl-4-chloro-1-[(2′-(1-H-tetrazol-5-yl)biphenyl-4-yl)methyl]-imidazole-5-carboxylic acid monohydrate Form VI is characterized by a principal d-spacing of 15.91 Å, more specifically 15.91, 9.99, 8.37, and 7.59 Å.

### Example 9

### Preparation of 2-butyl-4-chloro-1-[(2′-(1-H-tetrazol-5-yl)biphenyl-4-yl)methyl]-imidazole-5-carboxylic Acid Monohydrate Form VII

50 mgs of Form VI formed in Example 7 was slurried in approximately 0.5 mLs of water in a centrifuge tube. The samples were allowed to rotate on a rotator for approximately 72 hours. After 72 hours, the samples were centrifuged and the supernatant was removed. The residual solids were vacuum-dried (40° C.) for 24 hours. The solid was then analyzed by X-ray Powder Diffraction. The diffractogram for 2-butyl-4-chloro-1-[(2′-(1-H-tetrazol-5-yl)biphenyl-4-yl)methyl]-imidazole-5-carboxylic acid monohydrate Form VII is characterized by principal d-spacings of 9.13, 8.09, 6.61, 4.18, 3.70 and 3.65 Å, more specifically 10.99, 9.13, 8.69, 8.09, 6.61, 6.32, 6.24, 5.39, 4.37, 4.23, 4.18, 3.96, 3.93, 3.80, 3.70, 3.65, 3.24, 3.17 and 3.13 Å.

### Example 10

### Multiparticulates Based on Hydrophilic Swelling Polymers

Controlled release multiparticulates based on hydrophilic swelling polymers HPMC and poly(ethylene oxide) control the release of the drug embedded in the matrix. These controlled release multiparticulates may be combined with immediate release granules described below in various proportions in order to get the desired release profile between 4 and 6 hours.

### Controlled Release Multiparticulate Formulations 10A 10B and 10C

| Ingredient | 10A | 10B | 10C | % range w/w |
|---|---|---|---|---|
| Active Ingredient | 25 | 25 | 25 | 1-75 |
| HPMC (Methocel K4M) | 10 | 25 | — | 2.5-90 |
| Poly(ethylene oxide) | 10 | 25 | — | 2.5-90 |
| (Polyox WSR N60K) | | | | |
| HPMC (Methocel K15M) | — | — | 15 | 2.5-90 |
| Poly(ethylene oxide) | — | — | 15 | 2.5-90 |
| (Polyox WSR Coagulant) | | | | |
| Dicalcium phosphate | 27 | 12 | 22 | 2.5-90 |
| (ATAB) | | | | |
| Avicel PH 102 | 27 | 12 | 22 | 2.5-90 |
| (Microcrystalline cellulose) | | | | |
| Magnesium stearate | 1 | 1 | 1 | 0.5-5 |

Amounts are in % w/w.

### Procedure for Preparing Controlled Release Multiparticulate Formulations

The ingredients are accurately weighed and mixed in a Turbula mixer for 5 minutes. The blend was compressed into 2 g slugs using 1″ round flat tooling on automatic Carver press at 4000 kg force (77 MPa). The slugs were manually broken and sized into 500-180 um using sieves #35 and #80. The above can be commercially manufactured using roller compaction or wet granulation.

### Immediate Release Granules Formulation 10D

| Ingredient | 10D |
|---|---|
| Active Ingredient | 25 |
| Lactose | 35.5 |
| Avicel PH 102 (Microcrystalline cellulose) | 35.5 |
| Croscarmellose sodium | 3 |
| Magnesium stearate | 1 |

Amounts are in % w/w.

### Procedure for Preparing Immediate Release Granule Formulations

The ingredients are accurately weighed and mixed in Turbula mixer for 5 minutes. The blend was compressed into 2 g slugs using 1″ round flat tooling on automatic Carver press at 4000 kg force (77 Mpa). The slugs are then comilled into granules.

The above can be commercially manufactured using roller compaction or wet granulation.

### Combination of Controlled Release Multiparticulates and Immediate Release Granules

The controlled release multiparticulates are filled into capsules alone or in combination with immediate release granules to get a formulation with the desired release rate. The release of the drug depends on the drug/polymer ratio total polymer content, the grade of the polymers (high molecular weight vs. low molecular weight), the ratio of HPMC and polyethenyloxid Polyox. In addition, the release rate also depends on the ratio of the amount of controlled release multiparticulates to the amount of immediate release granules.

Alternative procedures to those described above use the roller compaction process in place of slugging, followed by milling to get multiparticluates and granules in the desired size range.

### Example 11

### Erodible Matrix Tablet

HPMC K4M is a swelling as well as erodible polymer that is used to control the release rate. Poloxamer 407 is added as a solubilizer in the formulation. In addition it is hypothesized that it can also modulate the erosion of the tablets.

### Erodible Matrix Tablet Formulations 11A, 11B and 11C

| Ingredient | 11A | 11B | 11C |
|---|---|---|---|
| Active Ingredient | 25 | 25 | 25 |
| HPMC (Methocel K) | 2.5 | 5 | 7.5 |
| Poloxamer 407 | 12 | 12 | 12 |
| Dicalcium phosphate (ATAB) | 27 | 27 | 27 |
| Avicel PH 102 (Microcrystalline cellulose) | 29.5 | 27 | 24.5 |
| Fast flo lactose | 3 | 3 | 3 |
| Magnesium stearate | 1 | 1 | 1 |

Amounts are in % w/w.

### Procedure for Preparing Erodible Matrix Tablet Formulations

The ingredients are dry mixed in a Turbula mixer, and 200 mg tablets are compressed in an automated Carver press using 10/32″ standard round concave tooling. Drug release was controlled by the HPMC level in the formulation. The formulation with 2.5% HPMC level released the drug in the desired 6 hrs.

The process of manufacture for this formulation can be direct compression, roller compaction, high shear wet granulation or fluid bed granulation.

### Example 12

### Coated Multiparticulate System

The controlled release multiparticulate system consists of drug granules (prepared by high shear wet granulation and spheronization techniques at 15% w/w drug loading) coated with an ethylcellulose aqueous dispersion (Aquacoat®) as a rate controlling barrier, using fluid bed coating.

### Coated Granule Formulation 12A

Coated granule formulations include a dry granule component and a coating component.

### Granule Component Formulation

| Ingredient | % w/w |
|---|---|
| Active Ingredient | 15 |
| Dibasic sodium phosphate, heptahydrate | 15 |
| Avicel RC 591 (Microcrystalline cellulose | 20 |
| with Sodium carboxymethylcellulose) | |
| Hydroxypropyl cellulose EXF grade | 2 |
| Avicel PH 101 (Macrocrystalline cellulose) | 48 |

Amounts are in % w/w.

### Granule Component Formation Procedure

Specified amounts of E3174, dibasic sodium phosphate heptahydrate, Avicel RC 591, Avicel PH 101 and half of the specified amount of hydroxypropyl cellulose were blended in a Turbula blender. The contents were transferred to a Bohle high shear granulator and granulated with a 10% w/v HPC EXF solution. The granulated mass was then spheronized on a Caleva spheronizer (Model 120) at an intermediate speed for 3 mts. and dried in a tray drier at 35° C. for a period of 14 hrs. The dried granules were then sized to yield granules in the particles size range of 180 to 355 μm.

### Coating Component Formulation

| Ingredient | % w/w solids basis |
|---|---|
| Aquacot ® | 68.8 |
| triethyl citrate | 24.1 |
| kaolin | 6.88 |
| FD&C blue lake | 0.28 |

The above formulation list amounts of ingredients on a weight percent solids basis. The coating solution is prepared in water at a 20% solids level.

### Coating Component Formation and Coating Procedure

The sized granules were transferred to a Miniglatt fluid bed coater and coated with an Aquacot® dispersion with 20% w/w solids content to achieve a weight gain of 8 percent. The Aquacoat dispersion also contains triethyl citrate as a plasticizer, kaolin and an FD&C blue lake at 35% w/w, 10% w/w and 0.4% w/w of solid ethyl cellulose, respectively. A top spray configuration was used for coating and a pneumatic vibrator was used for mitigating static effects. The coated granules were cured at 60° C. in a tray drier for 4 hrs and coated granules equivalent to 50 mg of the free base were encapsulated in a size 00 capsule. These coated multiparticulates may alternatively be lightly compressed in the form of tablets with use of cushioning brads for protecting the rate controlling polymer coat.

### Example 13

### Dissolution Profile

Formulations prepared in the above examples were evaluated in a USP dissolution apparatus II (paddle method) at 100 rpm in pH 6.8 phosphate buffer, with samples taken over a 14 hour period of time, e.g., 0.5, 1, 2, 3, 4, 6, 8, 10, 12 and 14 hours.

What is claimed is:

1. A polymorphic form of 2-butyl-4-chloro-1-[(2′-(1-H-tetrazol-5-yl)biphenyl-4-yl)methyl]-imidazole-5-carboxylic acid, or a pharmaceutically acceptable salt or hydrate thereof.

2. A polymorphic form of 2-butyl-4-chloro-1-[(2′-(1-H-tetrazol-5-yl)biphenyl-4-yl)methyl]-imidazole-5-carboxylic acid of claim 1 having X-ray powder diffraction pattern d-spacing readings selected from the group of readings consisting of
a) about 12.9, about 4.8, about 4.0, about 3.79, about 3.77, about 3.7, and about 3.4 Å,
b) about 8.6, about 6.5, about 5.7, about 3.9, and about 3.7 Å,
c) about 18.8, about 9.5, about 9.3, about 6.2, and about 4.2 Å,
d) about 8.6, about 5.0, about 3.7, and about 3.6 Å,
e) about 6.44, about 6.39, about 6.3, and about 4.2 Å,
f) about 7.4, about 6.8, and about 6.7 Å,
g) about 15.9 Å, and
h) about 9.1, about 8.1, about 6.6, about 4.2, about 3.7 and about 3.7 Å.

3. A polymorphic form of 2-butyl-4-chloro-1-[(2′-(1-H-tetrazol-5-yl)biphenyl-4-yl)methyl]-imidazole-5-carboxylic acid of claim 2 having X-ray powder diffraction pattern d-spacing readings selected from the group of readings consisting of
a) about 12.96, about 4.75, about 3.97, about 3.79, about 3.77, about 3.71, and about 3.44 Å,
b) about 8.55, about 6.54, about 5.68, about 3.90, and about 3.71 Å,
c) about 18.78, about 9.49, about 9.34, about 6.22, and about 4.20 Å,
d) about 8.57, about 5.01, about 3.66, and about 3.63 Å,
e) about 6.44, about 6.39, about 6.34, and about 4.20 Å,
f) about 7.38, about 6.75, and about 6.69 Å,
g) about 15.91 Å, and
h) about 9.13, about 8.09, about 6.61, about 4.18, about 3.70 and about 3.65 Å.

4. A polymorphic form of 2-butyl-4-chloro-1-[(2′-(1-H-tetrazol-5-yl)biphenyl-4-yl)methyl]-imidazole-5-carboxylic acid of claim 2 having X-ray powder diffraction pattern d-spacing readings selected from the group of readings consisting of
a) 12.96, 8.32, 8.13, 7.06, 5.18, 4.75, 4.64, 4.45, 4.41, 4.33, 4.19, 3.97, 3.86, 3.79, 3.77, 3.71, 3.59, 3.44, 3.15, and 2.92 Å,
b) 10.09, 8.55, 7.42, 7.26, 6.54, 6.43, 5.68, 4.39, 4.26, 4.17, 4.12, 4.10, 4.02, 3.90, 3.85, 3.71, 3.67, 3.63, 3.59, 3.44, 3.37, 3.35, 3.12, and 3.02 Å,
c) 18.78, 9.49, 9.34, 6.22, 6.18, 4.85, 4.67, 4.46, 4.20, 3.97, 3.68, 3.66, 3.63, and 3.50 Å,
d) 10.52, 8.57, 7.46, 6.60, 5.45, 5.37, 5.01, 4.91, 4.65, 3.80, 3.66, 3.63, 3.29, 3.23, 3.22, 3.19, 3.02 Å,
e) 7.34, 6.90, 6.44, 6.39, 6.34, 5.69, 5.64, 4.54, 4.26, 4.24, 4.20, 3.91, 3.90, 3.77, and 3.59 Å,
f) 7.38, 7.32, 6.75, 6.69, 4.38, 4.03, 3.76, 3.70, and 3.42 Å,
g) 15.91, 9.99, 8.37, and 7.59 Å, and
h) 10.99, 9.13, 8.69, 8.09, 6.61, 6.32, 6.24, 5.39, 4.37, 4.23, 4.18, 3.96, 3.93, 3.80, 3.70, 3.65, 3.24, 3.17 and 3.13 Å.

5. A polymorphic form of 2-butyl-4-chloro-1-[(2′-(1-H-tetrazol-5-yl)biphenyl-4-yl)methyl]-imidazole-5-carboxylic acid of claim 3 selected from the group consisting of 2-butyl-4-chloro-1-[(2′-(1-H-tetrazol-5-yl)biphenyl-4-yl)methyl]-imidazole-5-carboxylic acid hydrochloride Form I, 2-butyl-4-chloro-1-[(2′-(1-H-tetrazol-5-yl)biphenyl-4-yl)methyl]-imidazole-5-carboxylic acid hydrochloride Form II, 2-butyl-4-chloro-1-[(2′-(1-H-tetrazol-5-yl)biphenyl-4-yl)methyl]-imidazole-5-carboxylic acid hydrochloride Form III, and 2-butyl-4-chloro-1-[(2′-(1-H-tetrazol-5-yl)biphenyl-4-yl)methyl]-imidazole-5-carboxylic acid monohydrate Form IV, 2-butyl-4-chloro-1-[(2′-(1-H-tetrazol-5-yl)biphenyl-4-yl)methyl]-imidazole-5-carboxylic acid monohydrate Form V, 2-butyl-4-chloro-1-[(2′-(1-H-tetrazol-5-yl)biphenyl-4-yl)methyl]-imidazole-5-carboxylic acid monohydrate Form VI, 2-butyl-4-chloro-1-[(2′-(1-H-tetrazol-5-yl)biphenyl-4-yl)methyl]-imidazole-5-carboxylic acid monohydrate Form VII, and 2-butyl-4-chloro-1-[(2′-(1-H-tetrazol-5-yl)biphenyl-4-yl)methyl]-imidazole-5-carboxylic acid monohydrate Form VIII.

6. A pharmaceutical particle matrix composition comprising
a) an amount between about 1 and 75% w/w of crystalline 2-butyl-4-chloro-1-[(2′-(1-H-tetrazol-5-yl)biphenyl-4-yl)methyl]-imidazole-5-carboxylic acid of claim 2;
b) an amount between about 2.5 and 90% w/w of at least one water swellable polymer;
c) an amount between about 2.5 and 90% w/w of at least one filler; and
d) an amount between about 0.5 and 10% w/w of at least one lubricant,
wherein the particle size of the particle is between about 50 and 1200 μm.

7. A composition of claim 5, wherein the swellable polymer is preferably selected from the group consisting of polyethylene oxide and hydroxypropylmethyl cellulose, the filler is preferably a mixture of dicalcium phosphate and microcrystalline cellulose, and the lubricant is preferably magnesium stearate. water swellable polymer is selected from the group consisting of polyethylene oxide and hydroxypropylmethyl cellulose.

8. An erodible matrix composition pharmaceutical tablet comprising
a) an amount between about 1 and 75% w/w of crystalline 2-butyl-4-chloro-1-[(2′-(1-H-tetrazol-5-yl)biphenyl-4-yl)methyl]-imidazole-5-carboxylic acid of claim 2;
b) an amount between about 2.5 and 25% w/w of at least one water swellable polymer;
c) an amount between about 2.5 and 15% w/w of at least one solubilizing agent; and
d) an amount between about 2.5 and 90% w/w of at least one filler.

9. A composition of claim 7, wherein the water swellable polymer is selected from the group consisting of polyethylene oxide and hydroxypropylmethyl cellulose, the bulking agent is a mixture of dicalcium phosphate, microcrystalline cellulose and lactose, and the solubilizing agent is selected from the group of block copolymers of ethylene oxide and propylene oxide.

10. A pharmaceutical composition comprising a granule and a coating material coating the granule, wherein
a) the granule comprises
1) an amount between about 1 and 75% w/w of crystalline 2-butyl-4-chloro-1-[(2′-(1-H-tetrazol-5-yl)biphenyl-4-yl)methyl]-imidazole-5-carboxylic acid of claim 2;
2) an amount between about 5 and 50% w/w of at least one neutralizing agent;
3) an amount between about 1 and 25% w/v of at least one binder; and
4) an amount between about 5 and 75% w/w of at least one filler;
wherein the particle size of the granule is between about 100 and 1200 μm; and
b) the coating material comprises
1) a polymer deposited in the form of an aqueous dispersion or organic solution with subsequent evaporation of the dispersion solvents, wherein the amount of polymer in the coating material is such that the amount of polymer deposited on the granule is between about 5 and 40% w/w of the granule;
2) a plasticizer in the amount of up to 40% of the polymer weight; and
3) an anti-tacking agent in the amount of up to 10% of the polymer weight.

11. A composition of claim 9, wherein the neutralizing agent is dibasic sodium phosphate heptahydrate, the filler is microcrystalline cellulose, the binder is hydroxypropyl cellulose, and the aqueous dispersion comprises ethyl cellulose, triethyl citrate and kaolin.
